SEQUENCE LISTING (11 sequences)

SEQ ID NO: 1 - DNA, 1398 bp, Homo sapiens
atgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacaccagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 2 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETPVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 3 - DNA, 1395 bp, Mus sp.
atgatagtgtttgtcaggttcaactccagctatggcttcccagtggaggtcgattctgacaccagcatcttgcagctcaaggaagtggttgctaagcgacagggggttccagctgaccagctgcgtgtgatttttgccgggaaggagcttccgaatcacctgacggttcaaaactgtgacctggaacaacagagtattgtacacatagtacagagaccacggaggagaagtcatgaaacaaatgcatctggaggggacgaaccccagagcacctcagagggctccatatgggagtccaggagcttgacacgagtggacctgagcagccataccctgccggtggactctgtggggctggcggtcattctggacacagacagtaagagggattcagaagcagccagaggtccagttaaacccacctacaacagctttttcatctactgcaaaggcccctgccacaaggtccagcctggaaagctccgagttcagtgtggcacctgcaaacaagcaaccctcaccttggcccagggcccatcttgctgggacgatgtcttaattccaaaccggatgagtggtgagtgccagtctccagactgccctggaaccagagctgaatttttctttaaatgtggagcacacccaacctcagacaaggacacgtcggtagctttgaacctgatcaccagcaacaggcgcagcatcccttgcatagcgtgcacagatgtcaggagccctgtcctggtcttccagtgtaaccaccgtcacgtgatctgtttggactgtttccacttgtattgtgtcacaagactcaacgatcggcagtttgtccacgatgctcaacttggctactccctgccgtgtgtagctggctgtcccaactccctgattaaagagctccatcacttcaggatccttggagaagagcagtacactaggtaccagcagtatggggccgaggaatgcgtgctgcaaatgggaggtgtgctgtgcccccgtcctggctgtggagctggactgctacctgaacagggccagaggaaagtcacctgcgaagggggcaacggcctgggctgcgggtttgttttctgccgggactgtaaggaagcataccatgaaggggattgcgactcactgctcgaaccctcaggagccacttctcaggcctacagggtggacaaaagagccgctgagcaagctcgctgggaggaggcctccaaggaaaccatcaagaagaccaccaagccttgtcctcgctgcaacgtgccaattgaaaaaaacggaggatgtatgcacatgaagtgtcctcagccccagtgcaagctggagtggtgctggaactgtggctgtgagtggaaccgagcctgcatgggagatcactggtttgacgtgtag

SEQ ID NO: 4 - protein, 315 aa, Mus sp.
MVVRNSSYGVVDSDTSKVVAKRGVADRVAGKNHTVNCDSVHVRRRRSHTNASGGDSTSGSWSRSTRVDSSHTVDSVGAVDTDSKRDSAARGVKTYNSYCKGCHKVGKRVCGTCKATTAGSCWDDVNRMSGCSDCGTRAKCGAHTSDKDTSVANTSNRRSCACTDVRSVVCNHRHVCDCHYCVTRNDRVHDAGYSCVAGCNSKHHRGYTRYYGACVMGGVCRGCGAGGRKVTCGGNGGCGVCRDCKAYHGDCDSSGATSAYRVDKRAAARWASKTKKTTKCRCNVKNGGCMHMKCCKWCWNCGCWNRACMGDHWDV

SEQ ID NO: 5 - DNA, 1566 bp, Artificial
  Full length human Parkin fused to histidine tag
  misc_feature (14)..(29): His sequence
  misc_feature (157)..(168): Kozac sequence
atatacatatgcaccatcatcatcatcatttcttctggtctggtgccacgcggttctggtatgaaagaaaccgctgctgctaaattcgaacgccagcacatggacagcccagatctgggtaccgacgacgacgacaaggccatggctgatatcggatccgccgccaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacaccagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 6 - DNA, 720 bp, Artificial
  Green fluorescent protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 7 - protein, 239 aa, Artificial
  Green fluorescent protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 8 - DNA, 60 bp, Artificial
  Degradation signal (Degron)
gagatatcgcttgcaaagaactggttctcttccttgagtcacttcgtaattcacttgtag

SEQ ID NO: 9 - protein, 19 aa, Artificial
  Degradation signal (Degron)
EISLAKNWFSSLSHFVIHL

SEQ ID NO: 10 - protein, 1480 aa, Artificial
  CFTR protein
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILNEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECLFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 11 - protein, 3142 aa, Artificial
  Huntingtin protein
MATLEKLMKAFESLKSFQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

(19) United States
(12) Patent Application Publication
(10) Pub. No.: US 2007/0212679 A1
(43) Pub. Date: Sep. 13, 2007

(54) Assay for parkinson's disease therapeutics and enzymatically active parkin preparations useful therein

(75) Inventor: Jennifer A. Johnston, Mill Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Elan Pharmaceuticals, Inc., South San Francisco, CA (US)

(21) Appl. No.: 11/638,242

(22) Filed: Dec. 12, 2006

Related U.S. Application Data
(60) Provisional application No. 60/749,964, filed on Dec. 12, 2005.

(51) Int. Cl. C12Q 1/00; G01N 33/567

(52) U.S. Cl. 435/4; 435/7.2

(57) ABSTRACT

The invention provides to assays for agent useful for treatment of Parkinson's Disease. Included are cell-based assays for agents that modulate the effect of Parkin proteins on proteasome function. The invention also provides recombinant, enzymatically active, Parkin protein produced in prokaryotic expression systems, such as E. coli cells. Methods for purification of Parkin protein are also provided.

### FIELD OF THE INVENTION

This application claims benefit of U.S. provisional application No. 60/749,964 filed Dec. 12, 2005, the entire contents of which are incorporated herein by reference.

### FIELD OF THE INVENTION

The invention relates to assays for agent useful for treatment of Parkinson's Disease, as well as recombinant Parkin protein useful in assays. The invention finds application in the fields of protein purification, drug discovery, and medicine.

### BACKGROUND

Parkinson's disease (PD) is a neurological disorder characterized neuropathologically as a loss of dopamine neurons of the substantia nigra. This neuronal loss manifests clinically as alterations in movement, such as Bradykinesia, rigidity and/or tremor (Gelb et al., 1999, Arch. Neurol. 56: 33-39). Analysis of human genetic data has been used to characterize genes linked to the development of PD. One of these genes was localized to chromosome 6 using a cohort of juvenile onset patients and identified specifically as Parkin protein (Kitada et al., 1998, Nature 392: 605-608). Parkin protein has been shown to be an E3 ligase protein that functions in the ubiquitin-proteasome system (UPS) (Shimura, 2000, Nature Genetics 25:302-305). The UPS is a major cellular pathway involved in the targeted removal of proteins for degradation and E3 ligases function to identify and label substrates for degradation by cellular proteasomes (Hereshko and Cienchanover, 1998, Ann. Rev. Biochem. 67;425-479) or lysosomes (Hicke, 1999, Trends in Cell Biology 9:107-112).

A. Purification of Parkin-containing Inclusion Bodies

A sequence encoding full-length human Parkin fused to a histidine tag (see SEQ ID NO:5) was cloned into the bacterial expression plasmid pET30a (Novagen, Madison, Wis. 53719) to produce pET30a-Parkin. The N-terminal His tag is encoded by the pet30a vector and is fused in frame N-terminal to Parkin when a PCR fragment of Parkin (NM004562) with BamHI/HindIII restriction sites added to the 5′ and 3′ ends respectively is inserted into a pet30a vector also cut with BamHI/HindIII. E. coli. strain BL21(DE3)-pLysS were transformed and transformants were selected based on antibiotic resistance on LB plates with kanamycin.

10 ml overnight cultures of BL21 (DE3)-pLysS with pET30a-Parkin (grown in LB with 1% glucose, 25 ug/mL kanamycin and 35ug/mL chloramphenicol) were used to inoculate four flasks containing 1 liter each of LB +antibiotics. Cultures grew to an OD₆₀₀ of 0.55-0.6. To induce Parkin expression, IPTG was added to 0.4 mM and the flasks were returned to the shaker and grown at 37C for 4 hours. Cultures were collected by centrifugation (total pellet wet weight=15.6 g) and then frozen at −20C overnight.

The frozen pellets were resuspended in 140 mLs of Lysis Buffer (50 mM HEPES, pH 8.0, 500 mM NaCl, 1 mM EDTA, 10 mM beta-mercaptoethanol) and homogenized for 3 minutes to break up DNA. The resulting viscous solution was passed through a nebulizer 4 times. The resulting solution was cleared by centrifugation for 20 minutes at 30 k RCF (SS34 rotor) and the pellets (containing inclusion bodies) were recovered.

The inclusion bodies were suspended in 200 ml Wash Buffer #1 [50 mM HEPES, pH 8.0, 500 mM NaCl, 1% Triton X-100, 10 mM beta-ME] using the homogenizer for 2 minutes to disperse the suspension. The supernatant was saved for analysis and the inclusion bodies re-pelleted by centrifugation for 20 minutes at 30 k RCF.

The inclusion bodies were suspended in 200 ml Wash Buffer #2 [50 mM HEPES, pH 8.0, 1.0 M NaCl, 10 mM beta-ME], again using the homogenizer to disperse the solids. The supernatant was saved for later analysis and the inclusion bodies were repelleted by centrifugation for 20 minutes at 30 k RCF. The weight of inclusion body sample was 2.45 g.

The inclusion bodies were resuspended in 20 ml of Suspension Buffer (50 mM HEPES, pH 8.5, 6M GuHCl, 10 mM beta-ME) using the dounce homogenizer to break apart the solid mass. The sample, which was very dark brown in color, was left overnight at 4° C. The next morning, the remaining insoluble material was removed by centrifugation for 20 minutes at 30 k RCF and the supernatant was filtered through a 0.2 μM Tuffryn filter prior to use. 24.5 mis of supernatant was collected, with a protein concentration of 15.9 mg/ml.

B) Affinity Purification of Parkin Fusion Protein

The sample above was loaded onto a 40 mL IMAC column previously charged with nickel sulfate. The chromatography buffers were:

Buffers A: 50 mM HEPES, pH 8.0, 5.5M GuHCl, 10 mM beta-ME

Buffer B: 50 mM HEPES, pH 8.0, 5.5M GuHCl, 500 mM imidazole, 10 mM beta-ME pH was rechecked after all additions are made (except beta-ME, which was added fresh immediately prior to use.) The sample was loaded at 2 ml/min using 1% Buffer B with a total of 2 column volumes used to load the sample and wash the column. An additional wash at 4 ml/min using 5% B for column volumes 2.5 column volumes. 10 mL fractions were collected.

The sample was eluted at 4 ml/min using 2 column volumes of 100% Buffer B. 10 mL fractions were collected. As each column was collected additional beta-ME was added to 20 mM final concentration and 0.5M EDTA was added to 0.5 mM final concentration. Protein concentration was monitored during washing and elution and four 10-mL fractions collected during the elution step were pooled (“Pool 3”). The protein concentration of Pool 3 (50 mM HEPES, pH 8.0, 5.5M GuHCl, 500 mM imidazole, 20 mM beta-ME, 0.5 mM EDTA) was 2.22 mg/ml.

### EXAMPLE 8

Refolding Denatured Recombinant Parkin to Produce Active Enzyme

Pool 3 from Example 7 was diluted to a protein concentration of about 1.0 mg/ml with (50 mM HEPES, pH 8.0, 10 mM DTT) and 2×1 mL samples were dialyzed at 4C overnight against 500 mls (1.5M GuHCl, 50 mM HEPES, pH 8.0, 10 mM DTT) using 10 k MWCO dialysis tubing. No visible precipitation was evident the following morning. One of the samples was dialyzed overnight at 4C against (0.4M arginine, 50 mM HEPES, pH 8.0, 10 mM DTT). No apparent precipitation was apparent. The dialysate was collected and cleared by filtration.

Arginine was removed by further dialysis of the sample overnight at 4C. Sample 8B (500 uL at 1110 ug/mL) was made to ˜10% glycerol, then dialyzed against 1000 volumes 50 mM HEPES, pH 8.0, 0.2M NaCl, 10% glycerol, and 10 mM DTT. The following morning, no precipitate was visible in either sample. The samples were centrifuged for five minutes at top speed in a microfuge, then assayed for protein concentration. The recovery for Sample 8B was 78%.

### EXAMPLE 9

Optional SEC Purification

His₆-tagged Parkin was isolated from inclusion bodies as described in Example 6, section A. GuHCl-solubilized fractions were stored at −80° C. and quickly thawed and combined. Fresh DTT was added to 10 mM. Prior to loading on a 320 ml S200 chromatography column, the protein sample was concentrated using an Amicon Ultra15 with a 10 k MWCO. Final concentration was adjusted to 10 mgs/ml using SEC buffer (50 mM HEPES, pH 8.0, 3M GuHCl, 1 mM DTT (added fresh immediately prior to run)).

The column was equilibrated with 640 mls (2 CV's) of SEC Buffer at 1 ml/min (21° C.). 50 mgs of denatured His₆-Parkin (5 mls @ 10 mgs/ml) starting material was loaded onto the column at 0.75 mls/min. Flow was increased to 1.5 mls/min 174 mls into the run. 5 ml fractions were collected and additional DTT was added to 10 mM final. Fractions were stored at 4° C. until analyzed. When refolded as in Example 7, the resulting fraction (“#8BSEC”) had activity about the same the “#8B” material.

### EXAMPLE 10

Demonstration of Activity of Purified Parkin

An assay mixture containing His₆-Parkin prepared as described in Examples 6 and 7 was prepared. The 50 ul volume assay contained:

5 μM His₆-Parkin

100 nM human GST-E1 (Boston Biochem lot # 0271485, 7.35 μM)

5 μM UbcH7 (Boston Biochem lot # 1070224)

100 μM Ubiquitin*

50 mM HEPES pH 7.5

50 mM NaCl

1 mM Mg-ATP (omitted from controls)
*29.2 μl of 1 mM methylated ubiquitin (U-502, Boston Biochem lot# 2880574, dissolved in dH₂O to 1 mM) was used to resuspend 50 μg of biotin-ubiquitin (UB-560, Boston Biochem lot # 2011584). This results in 30μl of 1.17 mM ubiquitin with 17% biotinylated.

The reaction was incubated at 37° C. for. 0, 15, 30, 60 or 90 minutes, and reactions terminated by adding 6 μl 5× sample buffer (250 mM HEPES pH 7.5; 250 mM NaCl) plus 4 μl 1 M DTT.

A 15 μl aliquot of the assay mixture was electrophoresed on an 12% polyacrylaminde gel and transferred to a polyvinylidene fluoride (PVDF) membrane overnight (25V in 10 mM CAPS, pH 11, 10% MeOH, 4° C.) for Western blotting. The membrane was blocked 2 hours in TBST +5% BSA and incubated 1 hour at room temperature with NeutrAvidin-HRP (dilution: 1:7,500) in TBST+3% BSA (1 hour at room temperature). The membrane was washed 8×15 minutes with room temperature TBST. Uniform transfer from gel to membrane was confirmed by Ponceau S staining.

The results are shown in FIG. 7. Note that His₆-Parkin migrates at 57 kDa, UbcH7 at 18 kDa, Ub at 8.6 kDa, and His₆-Parkin-Ub complex at greater than 65 kDa. A distinct banding pattern was seen in the 66 kDa region starting at 15 minutes in reactions containing ATP. No ligase-dependent activity was observed in the absence of ATP at up to 90 minutes. Some signal was observed in the 66 kDa region when Parkin, E1, ubiquitin, and ATP were incubated for 90 minutes (lane 4).

The results shown in FIG. 7 are consistent with other experiments demonstrating ligase activity of E. coli produced Parkin, beginning at 15 minutes reaction time. The activity appears to plateau by 60 minutes-a further increase in product was not seen at 90 minutes. Generation of the distinct doublet product in the 60 kD region (as well as a high molecular weight smear) requires ATP; this eliminates the possibility that ubiquitin is simply co-aggregating with Parkin in a non-specific fashion. However, some reaction product was seen in the absence of UbcH7 when Parkin was incubated with E1 and ubiquitin for 90 minutes. This apparent ubiquitination of Parkin in the absence of UbcH7 is most likely a non-specific interaction between Parkin and E1. The amount of product generated at 90 minutes in this reaction was much less than the amount of product generated in the presence of UbcH7 at 15 minutes (compare lanes 4 and 12).

All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

There is an urgent need for new methods for treating Parkinson's disease. The present invention provides methods and materials that are useful for identifying and/or validating agents for PD therapy, as well as for other uses.

### BRIEF SUMMARY OF THE INVENTION

In one aspect, the invention provides assays for identification of, or screening for, compounds useful for treatment of Parkinson's Disease (PD). In one aspect, the invention provides a cell-based assay for identifying a candidate compound for treatment of Parkinson's Disease including (a) exposing a mammalian cell expressing Parkin to a test agent; and (b) comparing proteasome function in the cell with proteasome function characteristic of a corresponding mammalian cell expressing Parkin not exposed to the test compound; where an increased level of proteasome function in the cell exposed to the test agent indicates the agent is a candidate compound for treatment of Parkinson's Disease.

In a related aspect the invention provides a cell-based assay for identifying a candidate compound for treatment of Parkinson's Disease including(a) obtaining mammalian cells expressing Parkin; (b) exposing a cell to a test agent; and (c) comparing proteasome function in the cell with proteasome function in a cell not exposed to the test agent; where an increased level of proteasome function in the cell exposed to the test agent indicates the agent is a candidate compound for treatment of Parkinson's Disease.

Various methods may be used to measure or assess proteasome function. In one embodiment, the mammalian cells express GFPu and proteasome function is measured by measuring the amount of GFPu in the cells. In one embodiment the amount of GFPu in the cells is determined by measuring GFPu fluorescence.

In some cases the cell-based screening method also includes a proteasome function assay including (i) exposing a mammalian cell expressing a mutant Parkin to the candidate compound; and (ii) comparing proteasome function in the cell in with proteasome function characteristic of a cell expressing a mutant Parkin not exposed to the candidate compound.

In some cases the cell-based screening method also includes a proteasome function assay including (i) exposing a mammalian cell expressing another protein, such as Huntingtin, to the candidate compound; and (ii) comparing proteasome function in the cell with proteasome function characteristic of a cell expressing the other protein and not exposed to the candidate compound.

In some cases the cell-based screening method also includes an in vitro activity assay including (i) measuring the autoubiquitination activity of a purified Parkin protein in the presence of the compound; and (ii) comparing the autoubiquitination activity of purified Parkin protein in the presence of the compound with autoubiquitination activity of purified Parkin protein in the absence of the compound.

In some cases the cell-based screening method also includes an in vitro activity binding assay including (i) contacting the compound with purified Parkin protein and (ii) detecting the binding, if any, of the compound and the Parkin protein.

In another aspect, the invention provides purified Parkin protein and methods of obtaining such protein. In one aspect, the invention provides a method of purification of histidine tagged Parkin from inclusion bodies of bacterial cells expressing Parkin by (a) disrupting the inclusion bodies in the presence of guanidine-HCl and recovering a soluble fraction containing histidine tagged Parkin; (b) purifying the histidine tagged Parkin by affinity chromatography of the histidine tagged Parkin, in which the chromatography includes eluting bound protein with a solution comprising guanidium, thereby producing a composition containing histidine tagged Parkin and guanidium; (c) dialyzing the composition containing histidine tagged Parkin and guanidium against a buffered aqueous solution containing a high-concentration of arginine and a reducing agent to produce a first dialysate; and (d) dialyzing the first dialysate against a buffered aqueous solution substantially free of arginine. In one embodiment of the method, guanidine hydrochloride is used, optionally at a concentration of from 2 M to 6 M. In one embodiment of the method, guanadinium isothiocyanate is used. In some embodiments of the method the reducing agent is beta-mercaptoethanol, DTT or TCEP. In some embodiments of the method the high concentration of arginine in the buffered aqueous solution is from about 0.1 M to 1 M arginine. In some embodiments of the method the buffered aqueous solution substantially free of arginine contains less than 0.5 mM arginine, such as less than 0.1 mM arginine.

In one embodiment, the elution solution is 50 mM HEPES, pH 8.0, 5.5M GuHCl, 500 mM imidazole, 10 mM beta-ME, 0.5 mM EDTA; the buffered aqueous solution in (c) is 0.4 M arginine, 50 mM HEPES, pH 8.0, 10 mM DTT; and buffered aqueous solution in (d) is 50 mM HEPES, pH 8.0, 0.2M NaCl, 10 mM DTT.

In an aspect, the invention provides purified recombinant Parkin from a bacterial expression system, such as E. coli. In one aspect, the invention provides enzymatically active purified recombinant Parkin comprising a histidine tag. In one aspect, the invention provides enzymatically active Parkin obtained from a bacterial expression system. Parkin activity can be demonstrated using any assay that measures an enzymatic activity and/or biological function of Parkin. The enzymatically active Parkin obtained from a bacterial expression system may include a histidine tag.

In an aspect, the invention provides enzymatically active Parkin obtained from a bacterial expression system that has a high specific activity, such as of at least about 0.1 Unit (U), at least about 0.2 U, at least about 0.25 U, or at least about 1 U/0.5 microgram Parkin protein (where a Unit is defined as the ability to transfer 50 ng ubiquitin to Parkin in 15 minutes in the presence of human GST-E1, UbcH7, ubiquitin and Mg-ATP; or, equivalently, one-quarter unit is the ability to transfer 25 ng ubiquitin to Parkin in 30 minutes). The enzymatically active Parkin obtained from a bacterial expression system may include a histidine tag.

### BRIEF DESCRIPTION OF THE FIGURES

FIG. 1 shows an immunoblot demonstrating that overexpression of Parkin results in impaired proteasome activity.

FIG. 2A-E shows epifluorescent and immunofluorscent images illustrating that expression of Parkin protein leads to stabilization and aggregation of other proteasome substrates such as GFPu.

FIG. 3 shows FACscan analysis of GFPu levels in cells expressing GFPu and transfected with a vector expression Parkin or a Parkin mutant (2ug DNA). The bar graph shows GFPu levels 2 days post transfection. Mutants 167, 212, 275 and 289 decreased proteasome activity above the wild-type Parkin (PKN).

FIG. 4 shows formation of GFPu aggresomes after expression of various Parkin mutant cDNAs in HEK293/GFPu cells. Cells were transfected with 2ug cDNA and five days later epifluorescence images of each sample were recorded using the same camera settings for each sample to reflect the level of fluorescence intensity. Fluorescence intensity is a direct measure of GFPu levels in the cells.

FIG. 5A-B shows the distribution of Parkin protein in normal and PD brains.

FIG. 6 shows fluorescent images of cells transfected with a vector control (FIG. 6A, left) or wild type Parkin or (FIG. 6A, right) and the average fluorescence intensities from the cells (FIG. 6B).

FIG. 7 shows the results of an activity assay for purified human Parkin from recombinant E. coli. The lanes on the immunoblot show corresponds to (1) MW markers; (2) E1, UbcH7, ubiquitin, time=90 minutes; (3) E1, UbcH7, ubiquitin, ATP, time=90 minutes; (4) E1, ubiquitin, Parkin, ATP, time=90 minutes; (5) UbcH7, ubiquitin, Parkin, ATP, time=90 minutes; (6) E1, UbcH7, ubiquitin, Parkin, time=0; (7) E1, UbcH7, ubiquitin, Parkin, time=15 minutes; (8) E1, UbcH7, ubiquitin, Parkin, time=30 minutes; (9) E1, UbcH7, ubiquitin, Parkin, time=60 minutes; (10) E1, UbcH7, ubiquitin, Parkin, time=90 minutes; (11) E1, UbcH7, ubiquitin, Parkin, ATP, time=0; (12) E1, UbcH7, ubiquitin, Parkin, ATP, time=15 minutes; (13) E1, UbcH7, ubiquitin, Parkin, ATP, time=30 minutes; (14) E1, UbcH7, ubiquitin, Parkin, ATP, time=60 minutes; (15) E1, UbcH7, ubiquitin, Parkin, ATP, time=90 minutes.

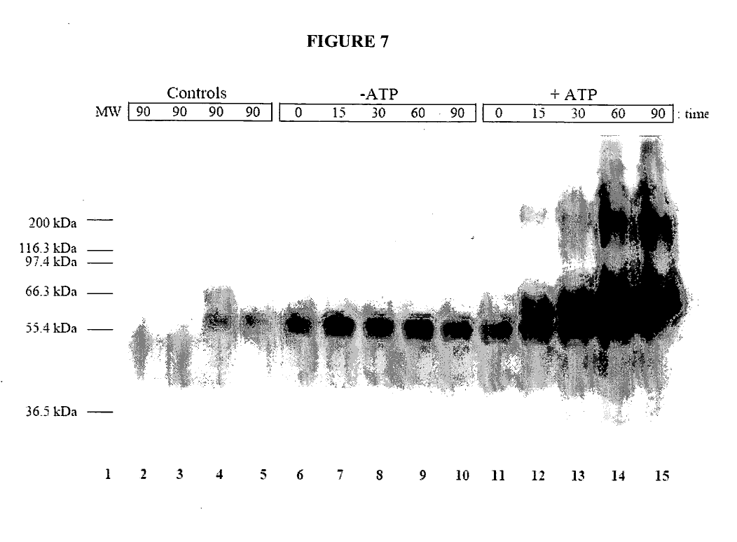

### DETAILED DESCRIPTION

I. Introduction

Genetic data have established that in humans loss of Parkin protein results in the progressive loss of dopaminergic neurons in the substantia nigra and eventually to Parkinson's Disease (“PD”). The relevant Parkin activity in dopaminergic neurons is likely to be its E3 ubiquitin ligase activity. The present invention contemplates a therapeutic approach to restore or augment Parkin ligase activity using therapeutic agents, such as small molecules, that can help Parkin achieve or maintain and active conformation. In one aspect, the invention relates methods for identification of agents useful for treating Parkinson's Disease. These methods include cell-based and protein-based assays.

In another aspect the invention provides methods for purification of enzymatically active Parkin expressed in recombinant bacterial cells. In a related aspect, the invention provides Parkin purified using these methods. The recombinant Parkin can be used in screening assays of the invention as well as for other applications (e.g., to establish standards for Elisa assays, for use as an immunogen to generate monoclonal antibodies, and other uses that will be apparent to scientists and physicians).

These and other aspects of the invention are discussed in more detail below.

II. Definitions

The terms “Parkin” and “Parkin protein” are used interchangeably and refer to wild-type Parkin or mutant Parkin.

“Wild-type Parkin” refers to human Parkin having the sequence of SEQ ID NO:2. or mouse Parkin having the sequence of SEQ ID NO:4. Wild-type Parkin can also refer to Parkin variants having mutation(s) that do not affect the ligase activity of Parkin and do not confer a different phenotype when expressed in cells. Sequences of nucleic acids and proteins encoding Parkin and other proteins are provided for the convenience of the reader and can also be found in the scientific literature. However, the practice of the invention is not limited to the specific sequences provides. It will be appreciated that variants also can be used in place of the sequences provided.

“Parkin mutant” or “mutant Parkin” refer to a Parkin protein with a sequence that deviates from SEQ ID NO:2 by a substitution, insertion or deletion of one or more residues, and has a different activity or confers a different phenotype than is conferred by wild-type Parkin. When referring to Parkin mutants, conventional nomenclature is used. For example, the R275W mutant has a substitution of tryptophan (W) for arginine (R) at position 275. Generally “Parkin mutant” refers to naturally occurring mutant proteins including, for example, R42P, S167N, C212Y, T240M, R275W, C289G, and P437L.

As used herein, reference to an “agent useful for treating Parkinson's Disease” or “candidate compound for treatment of Parkinson's disease” refers to a compound identified as being more likely than other compounds to exhibit therapeutic or prophylactic benefit for patients with Parkinson's disease, i.e., a drug candidate. It will be understood by those familiar with the process of drug discovery that a drug candidate may undergo further testing (e.g., in vivo testing in animals) prior to being administered to patients. It will also be understood that the therapeutic agent may be a derivative of, or a chemically modified form of, the drug candidate.

III. Identification of Agents Useful for Treating Parkinson's Disease

It has been discovered (see Examples below) that over-expression of wild-type Parkin in cells inhibits proteasome activity and can lead to the deposition of large insoluble inclusions of Parkin protein. Analysis of brain tissue showed that in PD patients Parkin levels appear to be elevated relative to healthy brain tissue and enriched in the insoluble fraction. See Example 5. Without intending to be bound by a particular mechanism, it is believed, based in part on experiments described in the Examples, that the wild-type Parkin protein is prone to misfolding, and that accumulation of misfolded Parkin results in: (1) impairment of proteasome activity, (2) generation of aggresomes containing Parkin and other cell proteins, (3) cell morbidity; and (4) loss of Parkin activity. Loss of Parkin activity is a direct mechanism leading to PD (Kitada et. al., 1998, Nature 392:605-608) and point mutations described in this work may also be related to the loss of function of Parkin leading to disease (Foroud et al., 2003, Ann. Neurology 60:796-801).

Moreover, it has been discovered that agents that stabilize Parkin (i.e., maintain Parkin in an active conformation even when over-expressed) or induce proper folding of misfolded Parkin are useful therapeutic agents for treatment of Parkinson's Disease. The present invention provides, inter alia, drug screening assays based, in part, on this discovery.

The invention provides both cell-based and protein based assays for such therapeutic agents.

A. Cell-Based Assays

As described in Examples 1-3, in cells in which wild type Parkin is over expressed, aggresomes (or “Parkin inclusions”) are formed and proteasome activity is decreased. In HEK293 cells in culture, Parkin protein is expressed endogenously at low levels. At this endogenous level of expression, the protein does not detectably affect the proteasome pathway, other than by performing its normal ligase activity. However, when Parkin protein is recombinantly expressed from a cDNA driven by an heterologous promoter (i.e., is expressed at high levels in cells compared to normal endogenous expression) Parkin protein, at least some of which is misfolded and/or insoluble, interferes with proteasome function. It is possible that the proteasome inhibition characteristics of high expression of Parkin levels of expression also occurs at a much lower, undetectable level under normal expression conditions. The slow accumulation of misfolded Parkin protein as an insoluble fraction in brain cells may occur over a long period (e.g., 40-80 years) leading to pathology.

Based in part on this discovery, the invention provides cell-based assays for identifying a candidate compound for treatment of Parkinson's Disease. In one assay of the invention, agents that stabilize Parkin or induce proper folding can be identified by the effect of the agent on aggresome formation and/or proteasome function in a cell.

In one embodiment, the assay includes screening for a candidate compound for treatment of Parkinson's Disease by (a) obtaining mammalian cells expressing Parkin; (b) exposing a cell to a test agent; and (c) comparing proteasome function in the cell exposed to the test agent with proteasome function in similar (control) cell not exposed to the test agent. An increased level of proteasome function in the cell exposed to the test agent compared to the control cell indicates the agent is a candidate compound for treatment of Parkinson's Disease. An exemplary assay is described in Example 6.

As discussed in more detail below, in various embodiments of this assay, the cells used may express wild-type Parkin or mutant Parkin. Preferably the level of expression is higher than the normal level for the particular cell used in the assay. In cases in which recombinant Parkin is expressed in a stably or transiently transfected cell, the expression level will essentially always be higher than normal. This is because endogenous levels of Parkin in cells are low and recombinant expression in which Parkin expression is driven by a heterologous (inducible or constitutive) promoter is comparatively high. Levels of Parkin expression in transfected or non-transfected cells can be measured using routine methods (e.g., immunostaining).

A.1 Proteasonie Function Assays

The effect of an agent on proteasome function in cells can be assessed using any assay of proteasome function. A primary proteasome function is degradation of intracellular proteins. In one embodiment of the assay, Parkin is expressed in a cell that also expresses a reporter-degron fusion protein, and the reporter is used to measure proteasome activity. The fusion protein includes a detectable polypeptide sequence with a degradation signal (“degron”) added to the C-terminus (or the N-terminus) of the protein. For illustration and not limitation, an exemplary degron sequence is provided as SEQ ID NO:9. The degradation signal serves to target the polypeptide to the proteasome where the polypeptide is degraded. When the activity of the proteasome is compromised, the levels of polypeptide in the cell increase relative to a cell in which the polypeptide is degraded by a normally functioning proteasome. An increase in protein levels can be detected in a variety of ways.

In one embodiment, proteasome function is assayed in cells using a GFPu reporter system. In the GFPu reporter system, cells that express a green florescent protein (GFP) with a degradation signal added to the C-terminus of the protein are used (see, Bence et al., 2001, Science 1552-55; Gilon et al., 1998, EMBO Journal 17:2759-66; SEQ ID NOS:6 and 9). As explained above, when the activity of the proteasome is compromised, the levels of GFP in the cell increase. An increase in GFP levels can be detected in a variety of ways including measuring GFP fluorescence levels in live cells or cell extracts and/or measuring levels of the GFU protein by ELISA, immunoblotting, and the like.

Other similar reporter systems can be used, for example, in which a reporter protein other than GFP is used and/or a different degron is used. See, for example, Dantuma et al., 2000, Nature Biotechnology 18:538-543. A variety of other proteins can be used, including reporter proteins such as Red Fluorescent Protein, Yellow Fluorescent Protein (e.g., Living Colors™ Fluorescent Proteins from Clontech, Mountain View Calif.), beta-galactosidase, luciferase, and the like. Alternatively, any polypeptide sequences detectable by virtue of an activity (e.g., an enzymatic activity that can be measured), antigenicity (e.g., detectable immunologically), a radioactive, chemoluminescent or fluorescent label, or the like. Degrons are known in the art (see, e.g., Gilon et al., 1998, EMBO Journal 17:2759-66; Sheng et al., 2002, EMBO J 21: 6061-71; Levy et al, 1999, Eur. J. Biochem. 259:244-52; and Suzuki and Varshavsky, 1999, EMBO J 18:6017-26).

In one version (“the basic assay”) the cell-based assay of the invention involves (a) transiently transfecting GFPu-expressing cells with an expression vector encoding wild-type Parkin (b) contacting a portion of the transfected cells with a test agent, and (c) determining whether the rate of degradation of the GFPu protein is increased, and GFPu levels are reduced in the cells contacted with the test agent compared to control cells not contacted with the test agent. Agents that reduce GFPu levels are candidates for further analysis and therapeutic use. It is expected that at least some agents that decrease GFPu levels do so by stabilizing Parkin structure, reducing the amount of misfolded Parkin.

Cell lines expressing the GFPu reporter are available from the ATCC (e.g., HEK-GFPu CRL-2794). Alternatively, cell lines expressing the GFPu reporter or other reporter-degron fusion proteins can be prepared de novo by transforming cells with a plasmid encoding the fusion protein. Any of a variety of cells can be used, including HEK293 cells (ATCC CRL-1573), SHSY-5Y cells (ATCC-2266), COS cells (CRL-1651); CHO cells (ATCC-CCL-61) or other mammalian cell lines. Cells can be stably or transiently transfected. Preferably the cells are stable transfectants for consistency across multiple assays.

In alternative embodiments, the assay can be carried out using cells stably expressing Parkin, and transiently transfected with the reporter-degron protein, or with cells transiently transfected with both Parkin and the reporter.

Expression vectors, methods for transient transfection, and methods for cell culture suitable for the practice of the invention are well known in the art and are only briefly described here. As is well known, expression vectors are recombinant polynucleotide constructs that typically include a eukaryotic expression control elements operably linked to the coding sequences (e.g., of Parkin). Expression control elements can include a promoter, ribosome binding sites, RNA splice sites, polyadenylation sites, and transcriptional terminator sequences. The expression vectors are typically replicable in the host organisms either as episomes or as an integral part of the host chromosomal DNA. Examples of mammalian expression vectors include pcDNA 3.1 (Invitrogen, San Diego, Calif.); pEAK (Edge Biosystems, Mountain View, Calif.); and others (see Ausubel et al., Current Protocols In Molecular Biology, Greene Publishing and Wiley-Interscience, New York, as supplemented through 2005). Commonly, expression vectors contain selection markers, e.g., ampicillin-resistance or hygromycin-resistance, to permit detection of those cells transformed with the desired DNA sequences. “Transfection” refers to a variety of art-recognized techniques for introducing foreign nucleic acid (e.g., DNA) into a host cell, including calcium phosphate or calcium chloride co-precipitation, DEAE-dextran-mediated transfection, lipofection, and electroporation. Cell culture techniques are also well known. For methods, see Sambrook et al. 1989, Molecular Cloning: A Laboratory Manual, 2nd Edition, Cold Spring Harbor Laboratory press; and in Ausubel, 1989, supra.

A.2 Cells Expressing Parkin

In a preferred proteasome function assay, cells expressing the reporter-degron fusion protein are transfected with an expression vector expressing Parkin, as described above. In one embodiment the expression vector encodes a wild-type Parkin. For example, the cDNA for human Parkin (NM004562) can be inserted into the HindIII/XbaI sites of the vector pcDNA3.1 (Invitrogen, San Diego Calif.) for use in this assay.

In another embodiment, an expression vector encoding a Parkin mutant is used. As shown in Example 4, expression of certain Parkin mutants results in inhibition of proteasome function. Proteasome function assays using such Parkin mutants can be conducted as described above for wild-type Parkin, except that cells are transfected with an expression vector encoding a Parkin mutant. This proteasome function assay involves exposing a mammalian cell expressing a mutant Parkin to the test compound; comparing proteasome function in the cell exposed to the test compound and proteasome function characteristic of a cell expressing the mutant Parkin not exposed to the test compound. Exemplary Parkin mutants include S167N, C212Y, T240M, R275W, C289G, P437L (see Table 1). In certain embodiments the Parkin mutant used is R275W, C212Y or C289G. Assays using Parkin mutants can be used as an alternative to, or in combination with, assays using wild-type Parkin.

TABLE 1
Six Parkin mutations for which heterozygosity
is correlated to development of PD

| Parkin mutation | Proposed mechanism of pathology |
|---|---|
| S167N | missense mutation/aggresome |
| C212Y | Dominant gain-of-function/aggresome |
| T240M | Loss-of-function |
| R275W | Loss-of-function |
| C289G | Reported to form aggresomes |
| P437L | missense mutation/aggresome |

A.3 Exposing Cell to Test Agents

As described above, Parken-expressing cells are exposed to a test agent to determine the effect of the agent on proteasome function. Most often, cells expressing a reporter fusion protein (see, e.g., Example 1) are grown and transfected with the Parkin encoding expression construct. The cells are cultured for 1-10 days and then exposed to a test agent. Usually the cells are exposed to a test agent 2 or 3 days after exposure to (or the beginning of exposure to) the agent.

A variety of classes of test agents can be used. For example, a number of natural and synthetic libraries of compounds can be used (see NCI Open Synthetic Compound Collection library, Bethesda, Md.; chemically synthesized libraries described in Fodor et al., 1991, Science 251:767-773; Medynski, 1994, BioTechnology 12:709-710; Ohlmeyer et al., 1993, Proc. Natl. Acad. Sci. USA 90:10922-10926; Erb et al., 1994, Proc. Natl. Acad. Sci. USA 91:11422-11426; Jayawickreme et al., 1994, Proc. Natl. Acad. Sci. USA 91:1614-1618; and Salmon et al., 1993, Proc. Natl. Acad. Sci. USA 90:11708-11712). In one embodiment, the agent is a small molecule, e.g., a “chemical chaperone,” such as a molecule with a molecular weight less than 1000, and often less than 500.

The duration of the exposure can vary, but will usually be from 1 to 24 hours, and most usually from 4 to 16 hours. Similarly, a variety of concentrations of agent can be tested. It will be appreciated that the concentration will vary depending on the nature of the agent, but is typically in the range of 1 nM to 5 uM. Typically several different concentrations of test agent are assayed (e.g., 1 nM, 10 nM, 100 nM, 1 μM, 10 μM and 100 μM) along with a zero concentration control.

The proteasome function of a Parkin-expressing cell contacted with test agent can be compared with proteasome function characteristic of a cell expressing Parkin but not exposed to the candidate compound. Typically this is accomplished by conducting parallel experiments using cells exposed to the test agent (at various concentrations) and cells not exposed to the test agent. That is, proteasome function in cells is measured in the presence or absence of compound. Alternatively, proteasome function in test cells can be compared to standard values obtained previously for proteasome function in cells. In another variation, proteasome function is measured in the same cells before and then after addition of the test agent.

At the end of culture period, proteasome function can be measured. For example, in GFPu-expressing cells, GFPu fluorescence and/or GFPu quantity can be measured. Measurements may be quantitative, semiquantitative and/or comparative.

It will be apparent to the reader that modifications of the basic assay can be made. For example, culture plates of various types (e.g., 6, 24, 96, or 384 well plates, ) or other high through-put devices can be used can be used for cell culture, optionally in combination with robotic devices, with concomitant adjustment of plasmid quantity in the transfection.

In one embodiment of the invention, HEK293 GFPu cells are grown to 75% density in culture wells of a six-well cell culture plate (e.g., each well approximately 30 mm in diameter). The cells are transfected the Parkin expression vector described above, using approximately 2.5 ug of plasmid per well, and the cells cultured for about 3 days (e.g., 2 to 5 days) prior to analysis with a test agent.

A.4 Proteasome Function Assays to Determine Whether The Effect of an Agent is Specific for Parkin

Proteasome function assays to establish Parkin specificity can be conducted by using the basic assay described above for wild-type Parkin, except that cells are transfected with an expression vector encoding a different protein believed to be prone to misfolding. For example, the Huntingtin (Htt) protein (SEQ ID NO: 11) or CFTR (SEQ ID NO: 10; accession number NM000492) proteins may be used. Other proteins prone to misfolding that may be used in this assay include SODI, Rhodopsin, connexin 43, Ub+1, and presenilin. This proteasome function assay involves exposing a mammalian cell expressing the non-Parkin protein (e.g., Huntingtin) to the candidate agent and comparing proteasome function in the cell with proteasome function characteristic of a cell expressing Huntingtin not exposed to the candidate agent. An agent that stabilizes or increases proteasome function in cells expressing Parkin but not cells expressing Huntingtin or other proteins is likely specifically modulating the effect of Parkin on proteasomes. An agent that stabilizes or increases proteasome function in cells expressing Huntinigtin or other proteins as well as in cells expressing Parkin may be acting nonspecifically.

A.5 Parkin Aggregation Assays

In one embodiment, the assay includes screening for a candidate compound for treatment of Parkinson's Disease by (a) obtaining mammalian cells expressing wild-type or mutant Parkin; (b) exposing a cell to a test agent; and (c) comparing Parkin aggregation in the cell exposed to the test agent with Parkin aggregation characteristic of a control cell not exposed to the test agent. A reduced level of Parkin aggregation in the presence of a test agent indicates the agent is a candidate compound for treatment of Parkinson's Disease. In one embodiment the mammalian cells express wild-type Parkin. In one embodiment the mammalian cells express a mutant Parkin. In some cases the mutant Parkin is S167N, C212Y, T240M, R275W, C289G, P437L. Preferably R275W, C212Y or C289G is used.

B. Parkin Binding Assays

An expected characteristic of many chemical chaparones is that they bind to the protein target. Thus, candidate agents useful for treatment of Parkinson's disease can be identified using a Parkin binding assay. The binding assays usually involve contacting purified Parkin protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet and Yamamura, 1985, “Neurotransmitter, Hormone or Drug Receptor Binding Methods,” in Neurotransmitter Receptor Binding (Yamamura, H. I., et al., eds.), pp. 61-89. The Parkin protein utilized in such assays can be from mammalian cells (recombinant or naturally occurring) or purified Parkin from recombinant bacterial cells.

C. Parkin Activity Assays

Parkin is an ubiquitin ligase (Shimura et. al., 2000, Nature Genetics 25:302). Ubiquitin ligase activity is defined by the ability of a protein to recognize a specific ligase substrate, and interact with an E2 enzyme to transfer an ubiquitin molecule from the E2 to the substrate. Ligase activity has been shown to be regulated by accessory proteins, but can also occur with the ligase alone (see Joazeiro and Weissman, 2000, Cell 102:549-52).

In one embodiment, an in vitro assay used to determine whether a candidate agent is useful for treating Parkinson's disease includes measuring the effect on Parkin ligase activity. In an embodiment the ligase activity is the autoubiquitination activity of a purified Parkin protein in the presence of the compound, and comparing the autoubiquitination activity of purified Parkin protein in the presence of the compound with autoubiquitination activity of purified Parkin protein in the absence of the compound. The ability of an agent to increase autoubiquitination activity is indicative of an agent useful for treating Parkinson's disease and a candidate for further testing. In addition, agents that stimulate autoubiquitination activity may increase the affinity of ligase for substrate, or prevent intracellular turnover of Parkin protein, and are therefore of interest for those activities as well.

Parkin autoubiquitination activity can be assayed in a solution assay or an immobilization assay, as described below and in the Examples.

C.1 Assays Using Immobilized Parkin

In the immobilization assay, recombinant or purified Parkin is immobilized on a surface (such as a microwell plate, sepharose beads, magnetic beads, and the like) and incubated with a ligase reaction mix that includes ubiquitin. The level of ubiquitination of Parkin under the assay conditions is determined as a measure of Parkin autoubiquitination activity.

Any method for immobilizing Parkin that does not interfere with Parkin activity can be used. In one embodiment, Parkin is immobilized in wells of a 96-well or 386-well microwell plate. Microwell plates are widely available, e.g., from Immulon (Waltham, Mass.) and Maxisorb (Life Technologies, Karsruhe, Germany). Parkin can be immobilized using an antibody binding system in which an antibody that recognizes Parkin is immobilized on a surface, and Parkin is added and captured by the antibody. Alternatively the antibody can recognize an epitope tag fused to the Parkin protein (e.g., His, GST, Flag, Myc, MBP, and the like). An antibody is selected that does not interfere with Parkin enzymatic activity. Methods for antibody-based immobilization and other immunoassays are well known (see, e.g., Harlow and Lane, 1988, Antibodies, A Laboratory Manual, Cold Spring Harbor Publications, New York). In an other approach, Parkin protein with a N-terminal His₆ tag can be immobilized using a nickel-coated assay plate.

After a blocking step, a ligase reaction mix including E1 (ubiquitin-activating enzyme, optionally epitope tagged, as with GST or His₆), E2 (ubiquitin conjugating enzyme), ATP-Mg, and ubiquitin (usually labeled ubiquitin) is combined with immobilized Parkin (Parkin E3 ligase). Purified ubiquitin pathway enzymes can be obtained commercially (e.g. from Boston Biochem Inc., 840 Memorial Drive, Cambridge, Mass. 02139) or prepared as described in Wee et.al., 2000, J. Protein Chemistry 19:489-498). Blocking to reduce nonspecific binding of E1 to the plate can be with SuperBlock (Pierce Chemical Company, Rockford, Ill.); SynBlock (Serotec, Raleigh, N.C. ); SeaBlock (CalBiochem, Darmstadt, Germany); metal chelate block (Pierce Chemical Company, Rockford, Ill. ); 1% casein; glutathione; and various combinations of these, with 1% casein preferred in some embodiments. After the blocking step, the wells can be washed with SuperBlock wash (Pierce Chemical Company, Rockford, Ill.) or Ligase buffer wash (50 mM HEPES/ 50 mM NaCl). In one embodiment, Immulon 96 or 384 well plates are blocked with 1% casein in 50 mM HEPES/50 mM NaCl and washed using 50 mM HEPES/50 mM NaCl/4mM DTT.

An exemplary reaction mix is:

| | |
|---|---|
| 1:1 Biotin:ubiquitin | 500 nM |
| GST-E1 | 2-6 nM |
| E2 (UbcH7) | 300 nM |
| E. coli Parkin protein | 2-10 ug |
| MgATP | 10 mM |
| Buffer | 50 mM HEPES/50 mM NaCl/pH 8.8 |

E. coli Parkin protein can be prepared as described below in Section IV. The assay can be carried out at 37° C. for 1 hour and stopped by washing wells with 50 mM HEPES/50 mM NaCl. ATP can be omitted from certain samples as a negative control. In one embodiment, the assay carried out in a 96 or 384 well plate format. The plate is incubated for a period of time (e.g., 60 minutes at room temperature or 40-60 minutes at 37° C.). Plates are washed to remove soluble reagents and the presence or amount of ubiquitin (i.e. the ubiquitin component of autoubiquinated Parkin) is determined.

Methods for detection of ubiquitin will depend on the label or tag used. For example, in a plate assay, fluorescein-tagged ubiquitin, can be detected directly using a fluorescence plate reader, biotin-tagged ubiquitin can be detected using labeled strepavidin (e.g., strepavidin-HRP or 1:5000 Neutravidin-HRP [Pierce Chemical Comp. Rockford, Ill.]), and epitope-tagged ubiquitin can be detected in an immunoassay using anti-tag antibodies. Epitope tags are fused to the N-terminus of ubiquitin or otherwise attached in a way the does not interfere with ubiquitination. These assays and other useful assays are well known the in art.

C2 Assays Using Parkin in Solution

In an alternative approach, the autoubiquitination assay for Parkin is carried out in solution and then the solution (or an aliquot) is transferred to a capture plate for quantitation. In an exemplary reaction, the reaction components (below) are assembled in 50 microliter volume and the assay is run for from 10 to 90 minutes (e.g., 60 minutes) at 37° C.

Reaction components:
50 mM HEPES/50 mM NaCl/pH 8.8
500 nM 1:1 Biotin: ubiquitin
2-6nM GST-E1
300 nM E2 (UbcH7)
2-10ug E. coli recombinant Parkin protein
10 mM MgATP*
*Can be added last to initiate the reaction.
   After reaction is complete, the reaction mix is transferred to a capture plate (e.g., 96 or 384 well plate) containing an immobilized moiety that binds Parkin (e.g., anti-Parkin antibody, nickel for His-tagged Parkin, or anti-epitope tagged antibody such as anti-flag GST, His, Myc, MBP, etc. for epitope-tagged Parkin). When nickel plates are used to immobilize His-tagged Parkin the reaction may be stopped by the addition of 6M Guanidinium HCl. This capture plate can be blocked with 1% casein. The reaction mix is incubated in the capture plate for 60 minutes. After this time, the plate is washed 3× with 50 mM HEPES/50 mM NaCl/4mM DTT). Detection is carried out using a reagent that binds to the tag present on the ubiquitin moiety (e.g., strepavidin-HRP) and processed using standard procedures.
   D. Combinations of Assays

The cell-based and protein-based assays described above can be used independently or in various combinations to identify candidate compounds for treatment of Parkinson's Disease that reduce proteasome impairment in cells expressing Parkin proteins. In one embodiment, the “basic assay” for agents that ameliorate the inhibition of proteasome function in cells expressing wild-type Parkin can be used in combination with additional assays such as: (1) proteasome function assays using Parkin mutants (2) Parkin aggregation assays (3) proteasome function assays to establish Parkin specificity (4) Parkin binding assays (5) in vitro protein activity assays. When used in combination, these assays can be conducted in any order. For example, initial high-throughput screening can be conducted using an in vitro protein assay and the basic cell-based assay can be used as a secondary screen. Alternatively, for example, cell-based assays can be conducted first and in vitro protein binding and activity assays can be used as a secondary screen. Other sequences and combinations of assays will be apparent to the reader.

In one embodiment agents that rescue proteasome function both in cells expressing wild-type Parkin and in cells expressing a mutant Parkin are identified as particularly promising drug candidates and subjected to further testing. In one embodiment agents are selected that rescue proteasome function in multiple cell lines, such as cells expressing proteins selected from wild-type Parkin and mutant Parkins (e.g., R275W, C212Y and C289G).

In one aspect of the invention, combinations of different cell-based assays and protein based assays are used to screen for agents useful for treatment of Parkinson's disease. For example, the basic cell based assay using wild-type Parkin can be using in conjunction with any one or combination of assays described above. Solely for illustration and not for limitation exemplary combinations of assays (and exemplary, non-limiting, profiles of agents considered useful) are shown in the table below. For example, one screening approach (C) comprises two assays: the cell based assay with wild-type Parkin and the Parkin activity assay. These assays can be conducted in any order.

TABLE 1

| | A | B | C | D | E | F |
|---|---|---|---|---|---|---|
| 1. Basic cell based assay with | + | + | + | + | + | + |
| wild-type Parkin | | | | | | |
| 2. Basic cell based assay with | + | − | | | + | |
| mutant Parkin | | | | | | |
| 3. Specificity assay | ++ | ++ | | | | ++ |
| 4. Protein binding assay | * | | | | | |
| 5. Protein activity assay | ** | | ** | | | |

+ Increases proteasome function
− Does not increase proteasome function
++ Does not increase proteasome function in nonParkin expressing cells
* binds
** increases ligase activity

IV. Expression and Purification of Enzymatically Active Recombinant Parkin

Wild-type and mutant Parkin proteins can be expressed and purified from recombinant mammalian cells (e.g., pcDNA-Parkin expressing vectors stably integrated into HEK-293 cells). Alternatively, recombinant Parkin can be obtained using Baculovirus expression or bacterial expression. However, heretofore, techniques that result in efficient purification of enzymatically active Parkin expressed in bacterial cells (e.g., E. coli, ) have not been described.

A. Expression of recombinant Parkin Protein

Parkin protein can be produced by expression in E. coli and other prokaryotic hosts using routine methods of transformation, selection, and culture. Exemplary E. coli strains that may be used include BL21; BL21 -pLysS; BL21-Star; BL21 -Codon+; BL21 (DE3); BL21(DE3)-Star; L21(DE3)-Codon+; and BL21-Al. Other useful bacterial expression systems include bacilli (such as Bacillus subtilus), other enterobacteriaceae (such as Salmonella, Serratia, Pseudomonas aeruginosa, and Pseudomonas putida) or other bacterial hosts (e.g., Streptococcus cremoris, Streptococcus lactis, Streptococcus thermophilus, Leuconostoc citrovorum, Leuconostoc mesenteroides, Lactobacillus acidophilus, Lactobacillus lactis, Bifidobacterium bifidum, Bifidobacteriu breve, Bifidobacterium longum, and Yersinia pestis).

Parkin can be expressed as a fusion protein with an affinity or epitope tag to facilitate purification. Exemplary tags include glutathione S-transferase (GST), dihydrofolate reductase (DHFR), maltose binding protein (MBP), 6× histidine [His]₆, chitin binding domain (CBD), and thioredoxin. A preferred tag is one that can be bound by an affinity ligand under denaturing conditions, e.g., in the presence of 6M guanidinium hydrochloride (GuHCl). A preferred tag is poly-histidine (e.g., [His]₆). Example 1 describes expression of full-length His-tagged Parkin for purification using nickel-mediated affinity chromatography.

B. Purification and Refolding of recombinant Parkin Protein

The inventors have discovered that when expressed in E. coli, Parkin partitions to inclusion bodies. Enzymatically active Parkin, particularly His₆-tagged Parkin, can be recovered from inclusion bodies of E. coli and other prokaryotes using a four-step process involving:

| | |
|---|---|
| 1) | Purification of inclusion bodies |
| 2) | Disruption of inclusion bodies |
| 3) | Chromatography |
| 4) | Refolding. |

Each of these steps is described below.

B.1 Purification of Inclusion Bodies

A variety of methods are known for purification of inclusion bodies and are suitable for practice of the present invention. See, e.g., Current Protocols in Protein Science (2003) Ch. 6: “Preparation and extraction of insoluble (inclusion-body) proteins from Escherichia coli.”

B.2 Disruption of Inclusion Bodies

A variety of methods are also known for disruption of inclusion bodies. See, e.g., Current Protocols in Protein Science, supra and Clark, 1998, Current Opinion in Biotechnology 9:157-163. In a preferred embodiment of the invention, inclusion bodies are disrupted using guanidine hydrochloride (e.g., 2 to 6 M GuHCl) and a reductant (e.g., 1 to 10 mM DTT; 4 mM TCEP [Tris (2-carboxyethyl)phosphine hydrochloride]; 4-10 mM beta-mercaptoethanol, and the like). For example, an inclusion body pellet can be combined with 5-10 volumes Suspension Buffer [50 mM HEPES, pH 8.5, 6M GuHCl, 10 mM beta-mercaptoethanol] and disrupted using a dounce homogenizer, sonication or other methods. Following disruption, any remaining insoluble material can be removed by centrifugation and/or filtration. The resulting soluble fraction contains Parkin and is suitable for affinity chromatography as described below.

In alternative embodiment, denaturants other than guanidinium hydrochloride can be used for disruption of inclusion bodies. In one alternative embodiment guanidinium isothiocyanate (e.g., 2-6 M) can be used. In other, less preferred, embodiments denaturants such as urea [2-8M]; sarkosyl( N-lauroylsarcosine) [1-2%]; Triton X-100+sarkosyl [0.5-2%+1-2%]; N-cetyl trimethylammonium chloride [2-5%]; N-octylglucoside [0.5-2%]; sodium dodecyl sulfate [0.1-0.5%]; alakaline pH to ph>9 (e.g., addition of NaOH); combinations of the foregoing; and other denaturants. See, Purification Handbook, Amersham Pharmacia Biotech p.71 (1999). Generally, the washed inclusion bodies are resuspended in the denaturants for 1-60 minutes (depending on the particular preparation, as well as the quantity of inclusion bodies being solubilized). It will be recognized that, in some cases (e.g., 8 M urea) the denaturant usually will be at least partially removed prior to affinity chromatography.

B.3 Affinity Chromatography.

The nature of the affinity chromatography used will depend on the tag used. As noted, the affinity interaction between the solid phase (affinity resin) and Parkin fusion protein should be stable in high concentrations of guanidinium hydrochloride (e.g., 2 to 6 M GuHCl).

In one embodiment, immobilized metal affinity chromatography (IMAC) is used. IMAC exploits the ability of the amino acid histidine to bind chelated transition metal ions, e.g., nickel (Ni²⁺), zinc (Zn²⁺), copper (Cu²⁺) or cobalt (Co²⁺). Usually nickel or cobalt is used. Immobilized nickel products for use in chromatography are readily available (e.g., Ni-NTA resins (Qiagen, Inc)). Immobilized cobalt products for use in chromatography are readily available (e.g., HIS-Select™ Cobalt Affinity Gel; Sigma-Aldrich Corp.).

Following application of the soluble fraction to the affinity column, the column is washed to remove unbound material and the Parkin-His₆ fusion protein is eluted. Conveniently fusion protein can be eluted using imidazole (e.g., 100-500 mM). In one embodiment, the elution buffer is 50 mM HEPES, pH 8.0, 5.5M GuHCl, 500 mM imidazole, 10 mM beta-ME, 0.5 mM EDTA. Fractions containing the target protein are recovered. Optionally, reductant can be added as fractions are collected (e.g. to increase the concentration of reductant can be increased the collected fractions (e.g., to 19 mM beta-ME).

B.3 Refolding

Two dialysis steps are used for recovery of active Parkin from the GuHCl-containing solution. In the first dialysis step, the eluted material is dialyzed against a buffered solution containing arginine and a reducing agent. Arginine may be present in the range 0.1 to 1 M, such as in the range 0.2 to 0.8 M, 0.3-0.6 M or 0.35-0.5 M). Exemplary reductants include DTT (e.g., 1 to 10 mM, e.g., 10 mM); Tris (2-carboxyethyl)phosphine hydrochloride (TCEP, e.g., 4 mM TCEP); beta-mercaptoethanol (e.g., 4-10 mM beta-mercaptoethanol), and agents with similar reducing power. An exemplary buffer is 0.4 M arginine, 50 mM HEPES, pH 8.0, 10 mM DTT.

In the second dialysis step the dialysate from step one is dialyze against a buffer that is substantially free of arginine. Any buffer in which proteins generally are stable (i.e., in which most proteins retain their structure and activity) can be used, so long as the buffer contains at most minimal amounts of arginine (i.e., less than 0.5 mM, preferably not more than 0.1 mM, most preferably no arginine). An exemplary buffer is 50 mM HEPES, pH 8.0, 0.2M NaCl, 10 mM DTT. Optionally, the glycerol can be added to the sample before this dialysis step. Additional dialysis steps can be carried out, if desired.

The dialysate containing Parkin can be collected and any precipitant removed. The activity of the purified protein can be determined using an autoubiquitination assay. See Lorick et al., 1999, “RING fingers mediate ubiquitin-conjugating enzyme (E2)-dependent ubiquitination” Proc Natl Acad Sci USA 96:11364-9 and Example 10. Preferably the specific activity of the purified Parkin material (>95% Parkin protein) is at least about 0.1 Unit/0.5 microgram Parkin protein. For example, the specific activity may be between 0.1 Unit per 0.5 micrograms and 5 Units/0.5 micrograms. In certain embodiments the specific activity is at least about 0.2 U, at least about 0.25 U, or at least 0.5 U/0.5 microgram Parkin protein In referring to specific activity a “Unit” is defined as the ability of a Parkin protein preparation to transfer 50 ng ubiquitin to Parkin in 15 minutes (e.g., under the assay conditions described in Example 10, below, where from 0.5 to 10 micrograms, usually 0.5 micrograms, Parkin protein is used in the reaction) or, equivalently, one-quarter unit is the ability to transfer 25 ng ubiquitin to Parkin in 30 minutes. His-tagged Parkin prepared according to the Examples, below, had a specific activity of approximately one-quarter Unit per 0.5 microgram Parken (i.e., 25.2 ng ubiquitin transferred in 30 minutes). Alternatively, Parkin activity can be demonstrated using any assay that measures an enzymatic activity and/or biological function of Parkin.

The purified Parkin protein can be used in a variety of applications, including screening assays, immunological assays, assay standards and others. The Parkin protein can be modified (e.g., conjugated to other compounds and/or an epitope tag removed) or processed as necessary for a particular application. In some embodiments the His epitope tag is removed. For example, the plasmid pET30a vector described in Example 7 the His-tag can be removed by digestion with thrombin or enterokinase (see SEQ ID NO:5).

### V. EXAMPLES

### Example 1

GFPu HEK293 Cells

GFPu-expressing cell lines were prepared as follows: HEK293 cells (ATCC No. CRL-1573) were transformed using a construct in which an oligonucleotide encoding a short degron (Gilon et al., 1998, EMBO Journal 17:2759-66) is inserted C-terminal to coding sequence for GFP (Heim et al., 1994, Proc. Nat. Acad. Sci. USA 91:12501-504; Accession #P42212). Cells were transfected with 2ug cDNA. The cells were cultured for 48 hours and transformants were selected using 1000 ug/ml G418 (geneticin). After an additional 7 days, the cell growth media (DMEM plus 1000ug/ml G418) was changed by removing old media and adding fresh media. Cells were allowed to grow for two weeks to select for cells that were resistant to G418. These cells were then collected and sorted by FACS techniques to identify and isolate single cells. These single cells were individually sorted into 96-well plates and allowed to grow and proliferate over two week period. The cells were then plated into duplicate 96 well plates. One plate was analyzed by FacScan the other plate was used to expand clones that were identified as positive in the FacScan analysis.

Clones were screened for very low background levels of GFP and an increase of more than 2 log units of fluorescence in the presence of the proteasome inhibitor epoxomicin. Two GFPu expressing cell lines, lines 60 and 61, were used in the remainder of the experiments.

Cells from the two GFPu cell lines were grown to 75% density in six-well plates, transfected with 2.5 ug per well of cDNA expression vectors encoding Parkin, Parkin mutants, Synuclein, or Synuclein variants. The cells were cultured for 2-5 days and examined using fluorescence microscopy and FACScan to measure GFP fluorescence. In some cases, epoxomicin was added 5 hours prior to FACScan as a positive control for GFPu levels. In addition, cell extracts were prepared for immunoblotting.

### Example 2

Expression of Wild-Type Parkin Results in Parkin Inclusions and Decreases in Proteasome Activity In GFPu-Expressing Cell Lines.

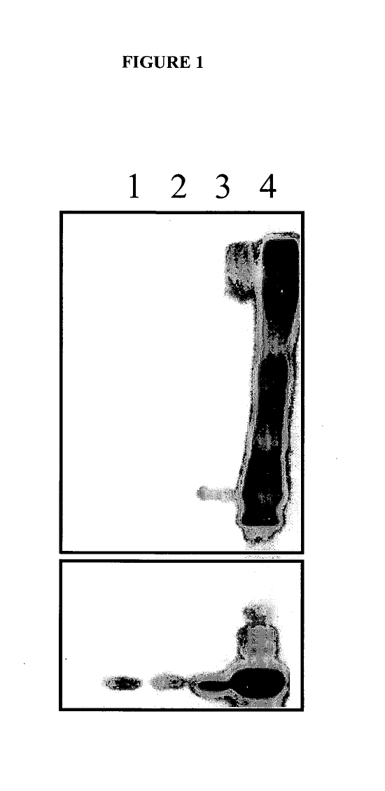

In both of the GFPu cell lines (lines 60 and 61), the overexpression of Parkin resulted in formation of Parkin aggregates, as determined by immunoblotting and microscopy. Parkin transfection also resulted in a striking increase in GFPu levels, indicating that expression or overexpression of Parkin impaired proteasome activity. FIG. 1 shows an immunoblot from cell line 60. GFPu/293 cells were transfected with pcDNA3.1 vector (lanes 1 and 2) or with pcDNA3.1-Parkin (lanes 3 & 4). 48 hours post transfection, cells were extracted for soluble protein and insoluble protein and these extracts were analyzed by immunoblotting for GFPu (bottom panel) or Parkin ( top panel). Soluble protein extract (lanes 1 and 3); insoluble protein extract (lanes 2 and 4). These data demonstrate a clear accumulation of GFPu protein after Parkin expression (compare lanes 3 & 4 with lanes 1 & 2), and also demonstrate the distribution of GFPu protein into the insoluble protein fraction after Parkin overexpression (compare lane 4 with lane 3).

### Example 3

Overexpression of Parkin, But Not Synuclein, Results in Aggresomes

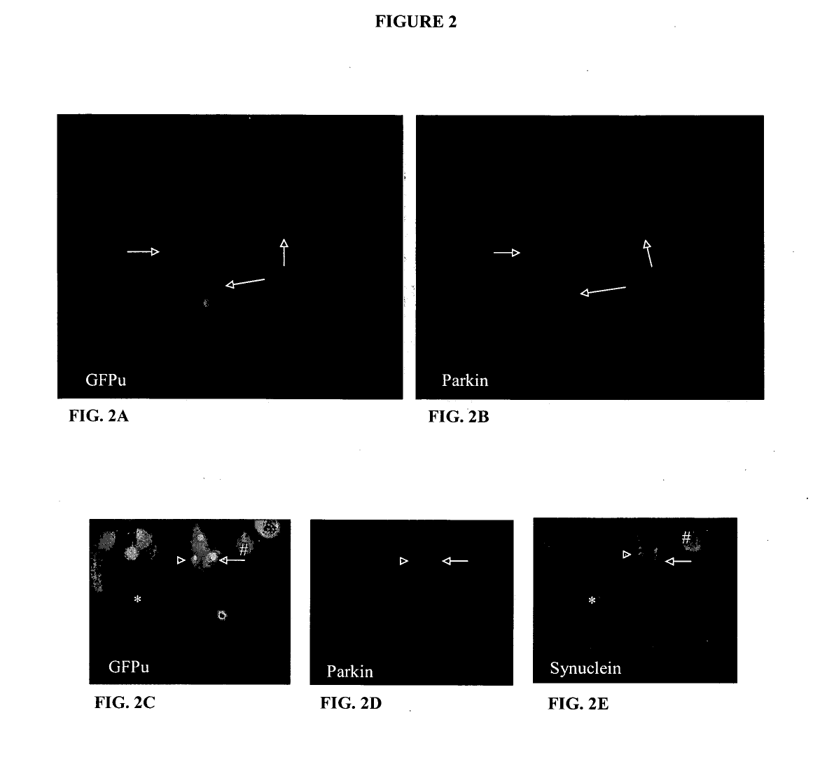

FIG. 2 shows epifluorescent and immunofluorscent images illustrating that expression of Parkin protein leads to stabilization and aggregation of other proteasome substrates such as GFPu. Parkin cDNA was transfected in to GFPu 293 cells prepared as described in Example 1 alone (Panels A and B) or with cDNA for alpha-synuclein and Parkin cDNA (Panels C, D and E). The cells were fixed after 48 hours and processed for immunofluorescence microscopy. Parkin protein was localized by staining with antibody HPA1A to residues 85-96 of human Parkin protein, alpha-synuclein was localized by staining with Syn-1 antibody (Transduction labs, San Jose, Calif.), and GFPu was localized based on the green fluorescence of the protein.

In cells expressing Parkin, Parkin protein (Panel A, arrows) is found as aggregates in the cells, and is colocalized with aggregates or accumulation of GFPu (Panel B, arrows). In cells not expressing Parkin protein (asterisk) there was no accumulation of GFPu.

In cells expressing both Parkin and alpha-synuclein, alpha-synuclein does not aggregate and is not required for the Parkin-mediated increase in GFPu. Arrows show that in cells expressing both synuclein and Parkin, aggregates of Parkin (Panel C) and GFPu (Panel D), but not of alpha-synuclein (Panel E), are found. Arrowhead indicates cells expressing only Parkin. The # symbol identifies a cell expressing alpha-synuclein but not Parkin. This cell does not have an increase in GFPu, indicating synuclein is not sufficient to increase GFPu. It is clear from this that the GFPu is accumulated/aggregated in cells expressing Parkin, and alpha-synuclein is not required.

### Example 4

Expression of Mutant Parkins Heterozygous “Dominant” Parkin Mutations

Expression plasmids encoding (1) wild-type Parkin or (2) mutant Parkin for which heterozygosity is correlated to development of PD were transfected into HEK 293/GFPu cells to assess the effect of the mutant Parkin proteins on proteasome function and aggregation (see Table 1).

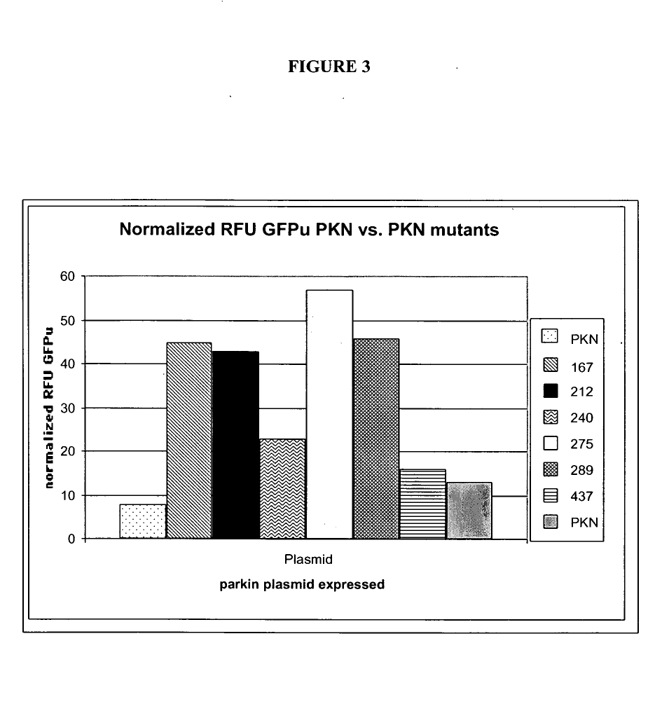

FIG. 3 shows the results of FACscan analysis 2 days post transfection. Inhibition of proteasome activity was significantly higher with mutants S167N, R275W, C212Y and C289G than for wild-type Parkin. Mutants R275W, C212Y and C289G significantly reduced proteasome activity at all times and transfection concentrations tested.

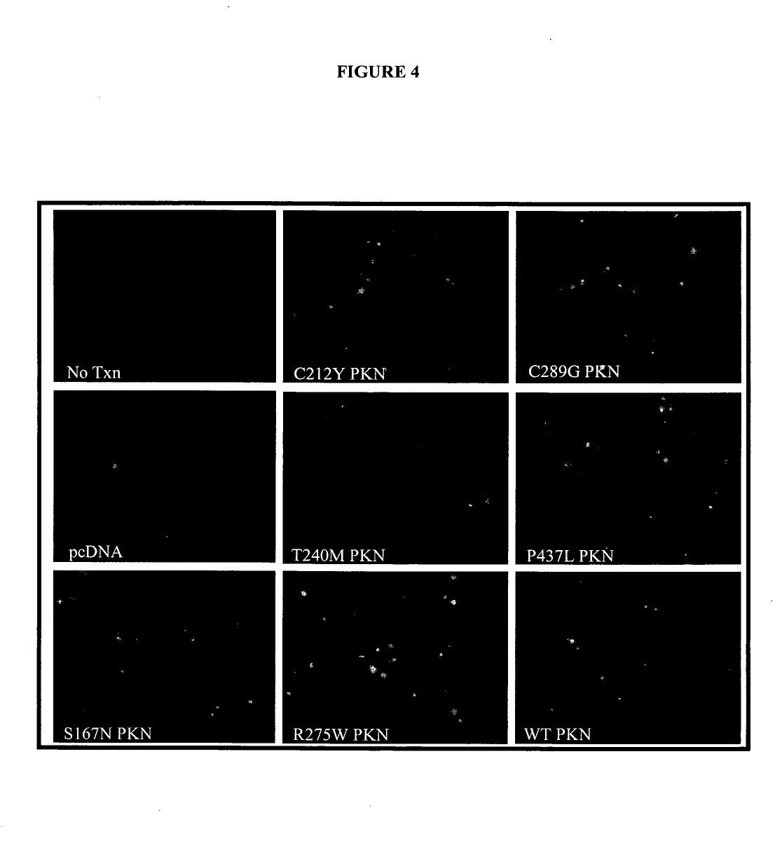

FIG. 4 shows epifluorescence images of each sample five days after transfection of the HEK 293/GFPu cells. The images were recorded using the same camera settings for each sample to reflect the level of fluorescence intensity, a direct measure of GFPu levels in the cells. As shown in the figure, expression of Parkin mutants can force GFPu into an aggresome. As shown in FIG. 4, and confirmed in experiments using an ArrayScan® high content screening device (data not shown), expression of mutants S167N, R275W, C212Y and C289G increased GFP levels (i.e., significantly reduced proteasome activity).

### Example 5

Parkin Distribution in Human Brain Tissue

The location and characteristics of Parkin protein in human brain tissue from sporadic PD patients and healthy controls was determined by immunoblotting of brain extracts.

Methods: Brain tissue from sporadic PD and normal individuals was obtained from the UCLA brain bank. Each sample consisted of tissue from four brain regions: Frontal cortex, caudate nucleus, putamen and substantia nigra. The later three brain regions are components of the nigrostriatal pathway. Frozen brain tissue from each brain region was homogenized via dounce, and extracted at a ratio of 0.5mg tissue/l ml of IPB extraction buffer (50 mM tris 7.5; 300 mM NaCl; 0.05% Deoxycholate; 0.1% NP-40, 5mM EDTA). After 20 minutes on ice, homogenates were spun for 10 minutes at 10,000×g. This supernatant was removed and the pellet was extracted again in the same manner, and centrifuged again. This second IPB supernatant was removed and the final remaining pellet was then sollubilized in 1% SDS/10 mM Tris 7.5 for ten minutes at room temperature, followed by sonication for 20 seconds.

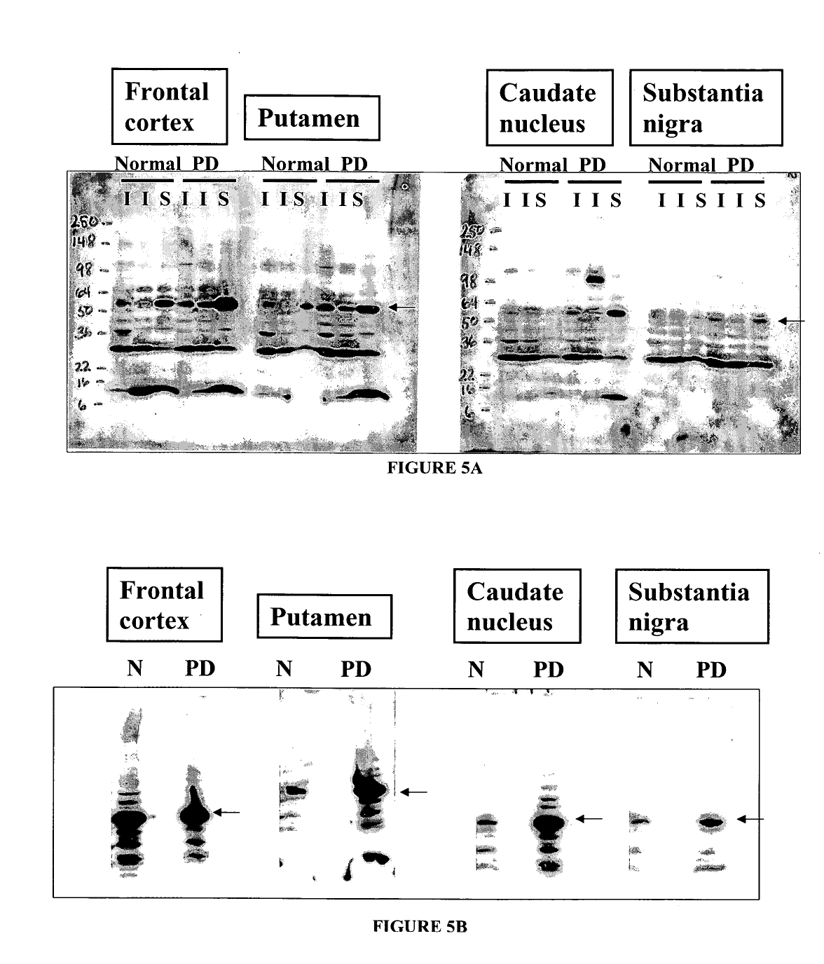

FIG. 5 shows the distribution of Parkin protein in normal and PD brain. Brain protein was seperated electrophoretically and immunoblotting was carried out using HPA1A, a polyclonal antibody to human Parkin residues 85-95. In FIG. 5A (I) is the IPB fraction and (S) is the SDS fraction, as described above. It is noteworthy that in the PD samples, the amount of 52-kD Parkin protein overall is increased, and the amount of insoluble Parkin is also increased.

A direct comparison of insoluble material from the same samples is provided in FIG. 5B. Compared to FIG. 5A, there is an accumulation of higher molecular weight material, possibly because the samples were sonicated just prior to loading the FIG. 5B gels, but not the FIG. 5A gels. Although Parkin is increased in the frontal cortex of both samples, it is increased in the nigrostriatal portions of the brain (putamen, caudate nucleus and the substantia nigra) only in the PD patients. The data in FIG. 5 suggest that in sporadic PD patients, Parkin levels may be increased relative to controls overall and enriched in the insoluble fraction. Insoluble protein is highly unlikely to be active.

### Example 6

Cell Based Assay

Hek293 cells stably expressing a proteasome-targeted Green Fluorescent Protein (GFP) were transiently transfected with an expression vector expressing wild type Parkin or with a vector only control (pEAK; Edge Biosystems, Mountain View, Calif.). The cells were maintained at 37° C. in 5% CO₂ for 16-24 hours. Cells were subsequently fixed with 3.7% formaldehyde and then washed 2× with PBS. Cells were then stained with 1 ug/ml Hoechst dye for 15 minutes at room temperature and then washed 2× with PBS leaving 200 ul of PBS in the well. Cells were imaged on the ArrayScan VTI using the XF100 filter set that had been optimized for GFP. Data were collected from at least 200 cells/well. The Target Activation Bioapplication program was used to analyze intracellular fluorescence (“mean average intensity”) where the mask modifier was set at 2 pixels.

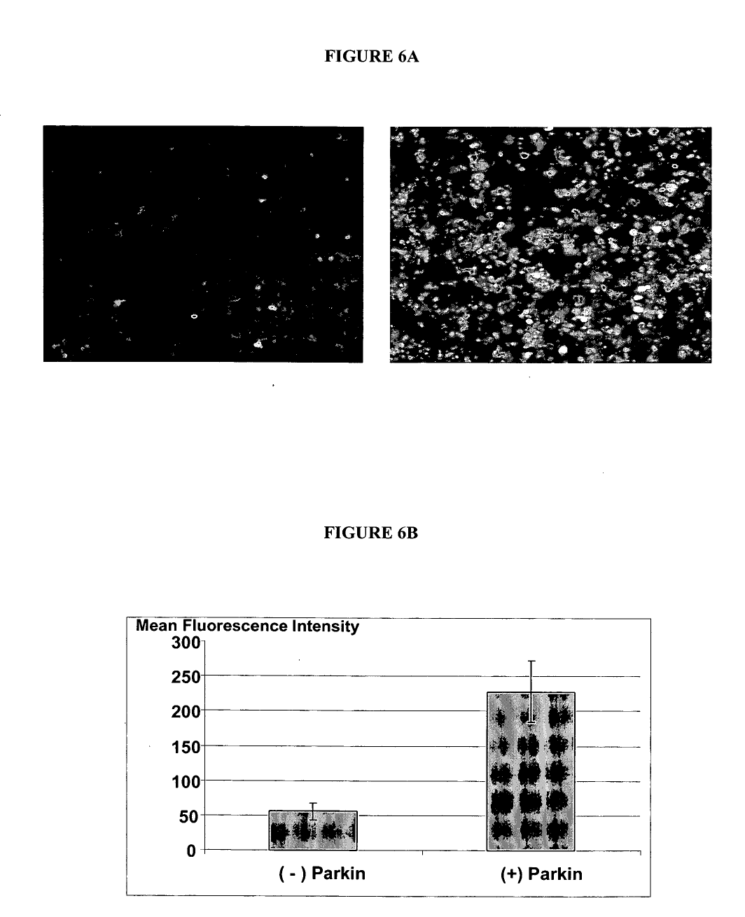

FIG. 6A shows fluorescent images of cells transfected with wild type Parkin (upper right) or vector control (upper left). In control transfected cells (vector alone) there was very little fluorescent intensity while in Parkin transfected cells exhibited a marked increase in GFP fluorescence intensity indicative of aggresome formation.

Similar results were observed in a number of different experiments. The signal to background ratio is consistently 3 to 5, where signal is defined as the mean fluorescence intensity from Parkin-transfected cells and background is mean fluorescence intensity from control treated cells (FIG. 6B). Because the cells are transiently transfected with DNA, we confirmed that there were not large variations in measured fluorescence intensities from well to well. Cells in each well of a 96-well plate were transfected with wild type Parkin and the mean average fluorescence intensity from each well was recorded. The coefficient of variation (CV) across the plate was quite low indicating the screening assay provides reliable consistent results.

A particular cell-based assay for identifying a candidate compound for treatment of Parkinson's Disease can be carried out as follows. Hek293 cells stably expressing a proteasome-targeted Green Fluorescent Protein (GFP) are obtained and are transiently transfected with an expression vector expressing wild type Parkin (“test cells”). Vector only control cells are also obtained. Four equivalent subcultures are prepared from the vector only cells and 16 test subcultures are obtained from the each parent culture. A test agent (“TA#100”) dissolved in culture medium. The test cells are provided with fresh culture medium containing 0, 1, 10, or 100 micrograms TA#100 and cultured under conditions in which Parkin is expressed. After 2 days the cells are fixed and processed as described above. The cells are imaged on the ArrayScan VTI using filters optimized for GFP. Data were collected from at least 200 cells/well. The fluorescence intensity and distribution in cells exposed to various amounts of TA#100 is determined, the fluorescence intensity being a measure of proteasome function in the cells. A decrease in fluorescence in the presence of TA#100 is evidence of an increased level of proteasome function in the cell exposed to the test agent and indicates the agent is a candidate compound for treatment of Parkinson's Disease. Additional assays are carried out to determine the dose-responsiveness of the effect. It will be appreciated that this example is for illustration and the reader guided by this specification will appreciate that there are numerous variations of this particular assay.

### Example 7

Expression and Purification of Recombinant Human Parkin

This example describes the expression and purification of a human Parkin-oligohistidine fusion protein (i.e., His₆-Parkin). Example 8 describes refolding denatured protein to obtain an enzymatically active product. Use of high concentration arginine and a strong reductant was effective in refolding denatured recombinant Parkin to produce active enzyme. Example 9 describes an optional additional chromatography step that may be used in purification.

1. A cell-based assay for identifying a candidate compound for treatment of Parkinson's Disease comprising
(a) exposing a mammalian cell expressing Parkin to a test agent;
(b) comparing proteasome function in the cell and proteasome function characteristic of a corresponding mammalian cell expressing Parkin not exposed to the test compound;
wherein an increased level of proteasome function in the cell exposed to the test agent indicates the agent is a candidate compound for treatment of Parkinson's Disease.

2. A cell-based assay for identifying a candidate compound for treatment of Parkinson's Disease comprising
(a) obtaining mammalian cells expressing Parkin;
(b) exposing a cell to a test agent;
(c) comparing proteasome function in the cell with proteasome function in a cell not exposed to the test agent;
wherein an increased level of proteasome function in the cell exposed to the test agent indicates the agent is a candidate compound for treatment of Parkinson's Disease.

3. The method of claim 1 wherein the mammalian cells express GFPu and proteasome function is measured by measuring the amount of GFPu in the cells.

4. The method of claim 3 wherein the amount of GFPu in the cells is determined by measuring GFPu fluorescence.

5. The cell based screening method of claim 1 further comprising
(a) a proteasome function assay comprising
(i) exposing a mammalian cell expressing a mutant Parkin to the candidate compound;
(ii) comparing proteasome function in the cell in (a)(i) and proteasome function characteristic of a cell expressing the mutant Parkin and not exposed to the candidate compound; and/or
(b) a proteasome function assay comprising
(i) exposing a mammalian cell expressing Huntingtin to the candidate compound;
(ii) comparing proteasome function in the cell in (b)(i) and proteasome function characteristic of a cell expressing Huntingtin not exposed to the candidate compound; and/or
(c) an in vitro activity assay comprising
(i) measuring the autoubiquitination activity of a purified Parkin protein in the presence of the compound; and
(ii) comparing the autoubiquitination activity of purified Parkin protein in the presence of the compound with autoubiquitination activity of purified Parkin protein in the absence of the compound; and/or
(d) an in vitro activity binding assay comprising
(i) contacting the compound with purified Parkin protein
(ii) detecting the binding, if any, of the compound and the Parkin protein.

6. The method of claim 5 that includes a proteasome function assay comprising (i) exposing a mammalian cell expressing a mutant Parkin to the candidate compound; (ii) comparing proteasome function in the cell and proteasome function characteristic of a cell expressing the mutant Parkin and not exposed to the candidate compound, wherein the mutant Parkin is R42P, S167N, C212Y, T240M, R275W, C289G, or P437L Parkin.

7. A method of purification of histidine tagged Parkin from inclusion bodies of bacterial cells expressing Parkin, said method comprising
(a) disrupting the inclusion bodies and recovering a soluble fraction containing histidine tagged Parkin;
(b) purifying the histidine tagged Parkin by affinity chromatography of the histidine tagged Parkin from (a), said chromatography comprising eluting bound protein with a solution comprising guanidine-HCl, thereby producing a composition comprising histidine tagged Parkin and guanidine-HCl;
(c) dialyzing the composition comprising histidine tagged Parkin and guanidine against a buffered aqueous solution containing a high-concentration of arginine and a reducing agent, thereby producing a first dialysate; and
(d) dialyzing the first dialysate against a buffered aqueous solution substantially free of arginine.

8. The method of claim 7 wherein the the inclusion bodies are disrupted in the presence of guanidine HCl or guanadinium isothiocyanate.

9. The method of claim 8 wherein the inclusion bodies are disrupted in the presence of 2 to 6 M guanidine hydrochloride.

10. The method of claim 7 wherein the reducing agent is beta-mercaptoethanol, DTT or TCEP.

11. The method of claim 7 wherein the high concentration of arginine in the buffered aqueous solution containing a high concentration of arginine contains from about 0.1 M to 1 M arginine.

12. The method of claim 7 wherein the buffered aqueous solution substantially free of arginine contains less than 0.5 mM arginine.

13. The method of claim 12 wherein the buffered aqueous solution substantially free of arginine contains less than 0.1 mM arginine.

14. The method of claim 7 comprising wherein the elution solution in (b) is 50 mM HEPES, pH 8.0, 5.5M GuHCl, 500 mM imidazole, 10 mM beta-ME, 0.5 mM EDTA; the buffered aqueous solution in (c) is 0.4 M arginine, 50 mM HEPES, pH 8.0, 10 mM DTT; and buffered aqueous solution in (d) is 50 mM HEPES, pH 8.0, 0.2M NaCl, 10 mM DTT.

15. A composition comprising enzymatically active purified recombinant Parkin comprising a histidine tag.

16. The composition of claim 15 wherein the Parkin is obtained from a bacterial expression system.

17. A composition comprising enzymatically active Parkin obtained from a bacterial expression system, said Parkin having a specific activity of at least about 1 Unit/0.5 microgram Parkin protein, when a Unit is defined as the ability to transfer 50 ng ubiquitin to Parkin in 15 minutes in the presence of human GST-E1, UbcH7, ubiquitin and Mg-ATP.

18. The composition of claim 17 wherein the Parkin comprises a histidine tag.
